(19) United States
(12) Patent Application Publication
(10) Pub. No.: US 2013/0288918 A1
(43) Pub. Date: Oct. 31, 2013

(54) Colorectal Cancer Screening Method

(71) Applicants: Jean-Pierre Roperch, Lommoye (FR); Iradj Sobhani, Saint Maur des Fosses (FR)

(72) Inventors: Jean-Pierre Roperch, Lommoye (FR); Iradj Sobhani, Saint Maur des Fosses (FR)

(73) Assignees: UNIVERSITE PARIS XII VAL DE MARNE, Creteil (FR); ASSISTANCE PUBLIQUE HOPITAUX DE PARIS, Paris (FR)

(21) Appl. No.: 13/883,435

(22) PCT Filed: Nov. 3, 2011
(86) PCT No.: PCT/FR2011/052571

§ 371 Date: Jul. 22, 2013

(30) Foreign Application Priority Data
Nov. 4, 2010 (FR) 10 59100

(51) Int. Cl. C12Q 1/68

(52) U.S. Cl. CPC C12Q 1/6886
USPC 506/9; 435/6.11

(57) ABSTRACT

The invention relates to a method of screening for colorectal cancer in a subject, comprising a step of measuring the degree of methylation of at least one of the genes selected from NPY, PENK and fragments or variants thereof in a biological sample obtained from said subject.

The present invention relates to a method of screening for colorectal cancer in a subject, comprising a step of measuring the degree of methylation of at least one of the genes selected from NPY, PENK and fragments or variants thereof in a biological sample obtained from said subject.

Colorectal cancer is a very widely occurring disease, which represents 10.5% of cancers world-wide, and is increasingly common (25000 new cases a year in the 1970s, 40000 new cases a year in the 2000s in France). Patients' life expectancy is still low, and is assessed at less than 50% at 5 years for all stages combined. It is a common, serious and expensive disease which in most countries is detected in systematic screening of persons at medium risk (asymptomatic, over 50 years of age).

The occult blood test is currently the most used test for screening for colorectal cancer. This test consists of testing for occult blood in the stool. Screening then comprises two steps: carrying out the occult blood test on three consecutive stool samples for detecting occult blood, followed, if the latter is positive, by a colonoscopy for detecting colorectal tumors. It is recognized that this test has two limitations: false positives (50%), leading to needless colonoscopies, and false negatives, so that colonoscopy is not carried out on patients with tumors. The degree of positivity of the test in the asymptomatic population in the age range 50 to 75 years is from 2 to 3%. Its positive predictive value, with reference to colonoscopy, is 50%.

Thus, in current screening conditions, nearly 50% of the colonoscopies performed are normal. However, nearly 50% of subjects with a colon tumor, benign or malignant, are not identified as such by this screening strategy.

This test (occult blood test) is open to criticism as it is not specific and is of low sensitivity. Moreover, because of reticence in manipulating feces, a proportion of the target population declines to undergo a screening test of this kind. Therefore there is now a need for a test that is specific, sensitive, and noninvasive, for detecting a subject's predisposition or risk of colorectal cancer.

Recent studies have highlighted the advantage of detecting methylation as a diagnostic or prognostic marker of various cancers. “Diagnosis” means determination that a person has a given pathology, and “prognosis” means the severity and subsequent development of a pathology. Methylation is a natural control mechanism that regulates DNA gene expression. For example, abnormal methylation of certain genes can decrease gene expression and may be associated with the development of cancers. It is now accepted that hypermethylation of the CpG islands on the promoters of tumor suppressor genes plays a fundamental role in the initiation and progression of cancers.

Patent application FR 2 927 091 describes a screening kit for colorectal cancer based on detection of the hypermethylation of a marker. However, this test is based on a single marker and does not have acceptable sensitivity.

Surprisingly, the inventors have successfully applied a molecular test for identifying several markers of interest whose hypermethylation is specific to colorectal cancers. After extensive research, the inventors have developed a method allowing DNA derived from intestinal tumor cells to be analyzed in biological effluents. It is thus possible to study the functional abnormalities of this DNA. This research has highlighted new markers specific to colorectal cancers.

The invention therefore relates to a molecular test based on the concept of methylation-specific PCR (MSP) in multiplex mode, or MSPM. MSPM has the major advantage of a decrease in the number of PCRs required relative to the monoplex mode. Thus, the multiplex mode offers a time saving, as it is quicker than several monoplexes, and is economically advantageous. Use of the MSPM technique also offers an undeniable advantage when the biological sample selected for application of the genetic test is blood, since the amount of DNA circulating in the blood is relatively small.

“Multiplex PCR” means a form of PCR, generally quantitative PCR, permitting simultaneous amplification of several targets of interest in a single step, using one or more specific primers. This technique is very advantageous for determining the presence of deletions, mutations, polymorphisms or hypermethylations of several markers.

In contrast, the expression “Monoplex PCR” refers to a form of PCR, generally a quantitative PCR, permitting amplification of a single target of interest.

“Methylation-specific PCR” means a conventional technique of the prior art for measuring the degree of methylation of a gene. This technique is based on the principle of quantitative PCR. Typically, this technique is based on treating the DNA sample to be investigated with sodium bisulfite. This treatment makes it possible to transform each of the unmethylated cytosines into uracils in the treated DNA. The sample thus treated then undergoes a PCR with primers specific to the genes to be treated. Determination of the nature of the specific primers depends on the nucleotide sequence to be amplified. In the context of this invention, methylation-specific PCR is preferably employed in multiplex mode, and is then called Methylation-Specific PCR in multiplex mode, or MSPM.

Thus, the invention relates to a method of screening for colorectal cancer in a subject, comprising a step of measuring the degree of methylation of at least one of the genes selected from the NPY, PENK genes and fragments or variants thereof in a biological sample obtained from said subject. “Screening for colorectal cancer” means detection of a predisposition to develop colorectal cancer, as well as detection of colorectal cancer already present in a subject.

“Methylation” means addition of a methyl group on carbon 5 of a cytosine in a CpG dinucleotide. These dinucleotides do not occur frequently in the structure of DNA, except in the CpG “islands”. These islands are typically represented at the level of the promoter region of the genes. Thus, when we talk of methylation of a gene, we are referring to methylation of the promoter region of said gene. The presence of a methyl group in a precise site prevents interaction between the gene and the transcription factors. Typically, the methyl groups prevent the transcription factors attaching to the amplification site and to the promoter, and prevent RNA polymerase attaching to the initiation site. Thus, methylation of the promoter region leads to repression of DNA transcription. The expression “methylation of a gene” encompasses methylation of the CpG islands of the nucleotide sequence of the gene but also methylation of the nucleotide sequences of the promoter of the gene to which said expression is applied.

“NPY gene” means the gene that codes for the neuropeptide Y or NPY.

“PENK gene” means the gene that codes for the neuropeptide proenkephalin A or PENK.

These two neuropeptides are involved in food intake, absorption of nutrients and equilibrium of the body's energy expenditure. They are neuropeptides that regulate the digestive functions, which can also regulate food intake and neuro-digestive behavior.

“Fragment of a gene” means a sequence of said gene with a length of at least 50 base pairs, preferably with a length of between 60 and 110 base pairs.

“Variant of a gene” means a nucleic acid sequence having a percentage identity of at least 80%, preferably of at least 90%, more preferably of at least 98% relative to said gene or to a fragment of said gene.

“Percentage identity” between two nucleic acid sequences or in the sense of the present invention denotes a percentage of nucleotides between the two sequences to be compared, obtained after optimal alignment, this percentage being purely statistical and the differences between the two sequences being distributed at random and over their entire length. “Best alignment” or “optimal alignment” denotes the alignment for which the percentage identity determined as described below is highest. Sequence comparisons between two nucleic acid sequences are traditionally performed by comparing these sequences after aligning them optimally, said comparison being carried out per segment or per “comparison window” for identifying and comparing the local regions with sequence similarity. Optimal alignment of the sequences for comparison can be done, besides manually, using the local homology algorithm of Smith and Waterman (1981), using the local homology algorithm of Neddleman and Wunsch (1970), using the similarity search method of Pearson and Lipman (1988), or by means of computer software using these algorithms (GAP, BESTFIT, BLAST P, BLAST N, FASTA and TFASTA in the Wisconsin Genetics Software Package, Genetics Computer Group, 575 Science Dr., Madison, Wis.). To obtain optimal alignment, preferably the BLAST program is used, with the BLOSUM 62 matrix.

The percentage identity between two nucleic acid sequences is determined by comparing these two sequences aligned optimally; the nucleic acid sequence to be compared can comprise additions or deletions relative to the reference sequence for an optimal alignment between these two sequences. The percentage identity is calculated by determining the number of identical positions for which the nucleotide is identical between the two sequences, dividing this number of identical positions by the total number of positions compared and multiplying the result obtained by 100 to obtain the percentage identity between these two sequences.

“Variants having a percentage identity of at least 80%, preferably of at least 90%, more preferably of at least 98%” denotes the nucleic acid sequences having, relative to the reference nucleic acid sequence, certain modifications such as in particular a deletion, a truncation, an elongation, a chimeric fusion, and/or a substitution, notably a point substitution, and whose nucleic acid sequence has at least 80%, preferably at least 90%, more preferably at least 98%, of identity after optimal alignment with the reference nucleic acid sequence.

In a particular embodiment, the method of screening for colorectal cancer according to the invention comprises a step of measuring the degree of methylation of at least one of the polynucleotide sequences selected from SEQ ID No. 1 and SEQ ID No. 2.

Sequence SEQ ID No. 1 corresponds to a fragment of the sequence of the NPY gene. As for sequence SEQ ID No. 2, it corresponds to a fragment of the sequence of the PENK gene.

“Subject” means an individual who is healthy or may have a cancer or who requires screening, diagnosis or monitoring.

“Biological sample” means a sample containing biological material. More preferably, it means any sample containing genetic material, such as nucleic acids. This sample can be derived from biological sampling performed on a living being (human patient, animal, plant). Preferably, the biological samples according to the invention are selected from all the fluids obtained from a human subject, for example whole blood, plasma, serum, lymph, urine, saliva, feces, or sweat.

“Nucleic acid material” means any sequence of deoxyribonucleic acid (DNA) or ribonucleic acid (RNA). Preferably, the biological sample is submitted to a step of extraction of the nucleic acid material before the step of measuring the degree of methylation. This extraction of the nucleic acid material is carried out according to the classical techniques, well known by a person skilled in the art. For example, this extraction can be carried out by cellular lysis of the cells obtained from the biological sample. This lysis can be of a chemical, physical, or thermal nature. This cellular lysis is generally followed by a purification step for separating and concentrating the nucleic acids from other cellular debris. Said extraction and said purification can be carried out using commercial kits of the QIAGEN and Zymo Research type. Of course, the relevant techniques differ depending on the nature of the biological sample tested. The knowledge of a person skilled in the art allows him easily to adapt these steps of lysis and purification to said biological sample being tested.

Preferably, the method according to the invention further comprises a step of measuring the degree of methylation of the Wif1 gene, one of the fragments or variants thereof in a biological sample obtained from said subject. In a particular embodiment, the method of screening for colorectal cancer according to the invention comprises a step of measuring the degree of methylation of the polynucleotide sequence SEQ ID No. 3. The sequence SEQ ID No. 3 corresponds to a fragment of the sequence of the Wif1 gene.

More preferably, measurement of the degree of methylation of the Wif1 gene or a fragment or variant thereof is carried out simultaneously with measurement of the degree of methylation of at least one of the genes selected from NPY, PENK and fragments or variants thereof in a biological sample obtained from said subject.

The Wif1 gene is an inhibitor of the Wnt pathway. The element that is the initiator of colorectal carcinogenesis is inactivation of the APC gene, the protein of which plays a role in connection with the control of proliferation, apoptosis and migration of the intestinal epithelial cells. This inactivation causes activation of certain signalling pathways, such as that of WNT/Beta-catenin and the formation of early adenomas. It is known that Wif1 is one of the genes involved in inhibition of the Wnt signalling pathway. Thus, hypermethylation of the Wif1 gene is accompanied by removal of inhibition of this pathway.

Preferably, the method according to the invention further comprises a step of measuring the degree of methylation of a housekeeping gene. Preferably, this step of measuring the degree of methylation of the housekeeping gene is carried out simultaneously with measurement of the degree of methylation of at least one of the genes selected from NPY, PENK and fragments or variants thereof and/or simultaneously with measurement of the degree of methylation of the Wif1 gene or a fragment or variant thereof.

In a particular embodiment, the method of screening for colorectal cancer according to the invention comprises a step of measuring the degree of methylation of the sequence SEQ ID No. 4. The sequence SEQ ID No. 4 corresponds to a fragment of the sequence of the albumin gene.

“Housekeeping gene”, or “constitutive gene”, means a constitutive gene that mainly codes for proteins that are essential for basic cellular functions. Preferably, expression of the housekeeping genes according to the invention is ubiquitous and stable among the various tissues. The housekeeping gene is preferably selected from the genes coding for albumin, beta actin, alpha actin, beta microglobulin, preferably albumin. These housekeeping genes are of major interest in the context of the invention. In fact, the degree of methylation of the NPY, PENK and Wif1 genes or fragments or variants thereof is expressed in relation to the methylation of a housekeeping gene whose expression is stable.

Typically, in the context of the present invention, measurement of the degree of methylation of the genes of interest is carried out according to the classical techniques of the prior art. Preferably, said measurement is carried out according to the “methylation-specific PCR” technique.

Typically, for amplification of the Wif1 gene, the sense primer SEQ ID No. 9 is used, said sequence having 19 bases and 3 CpG sites, and the antisense primer SEQ ID No. 10, said primer having 24 bases and 2 CpG sites. The TaqMan-MGB probe SEQ ID No. 11 has 16 bases and 2 CpG sites.

Typically, for amplification of the NPY gene, the sense primer SEQ ID No. 12 is used, said sequence having 20 bases and 3 CpG sites, and the antisense primer SEQ ID No. 13, said primer having 23 bases and 4 CpG sites. The TaqMan-MGB probe SEQ ID No. 14 has 18 bases and 3 CpG sites.

Typically, for amplification of the PENK gene, the sense primer SEQ ID No. 15 is used, said sequence having 21 bases and 3 CpG sites, and the antisense primer SEQ ID No. 16, said primer having 21 bases and 2 CpG sites. The TaqMan-MGB probe SEQ ID No. 17 has 18 bases and 3 CpG sites.

Typically, for amplification of the albumin gene, the sense primer SEQ ID No. 18 is used, said sequence having 22 bases and no CpG site, and the antisense primer SEQ ID No. 19, said primer having 22 bases and no CpG site. The TaqMan-MGB probe SEQ ID No. 20 has 17 bases and no CpG site.

“Degree of methylation” or “level of methylation” means the percentage of the number of methylated CpG sites of the target gene relative to a standard. Target gene means a gene for which we wish to measure the degree of methylation. In the context of the present invention, these target genes are Wif1, NPY, PENK, albumin and fragments or variants thereof. Standard means the wild-type sequence of said target gene or else the sequence of a housekeeping gene.

The degree of methylation is calculated by the quantification techniques that are well known by a person skilled in the art. This quantification can be absolute or relative. Preferably, it is calculated by the so-called ΔΔCt technique. This method employs an arithmetic formula for expressing the degree of methylation of a target gene, by normalizing with a reference gene. First, the differences ΔCt between the values of Ct of the target gene and of the reference gene are determined for the sample to be analyzed and the standard DNA. The standard DNA is typically a universally methylated DNA. It allows normalization of the degrees of methylation of the genes. A DNA of this kind is for example marketed by the company Zymo Research under the trade reference D5011. Following modification using a suitable kit, such as the commercial kit EZ DNA Methylation (ref. D5002, Zymo Research), this standard DNA is used as reference. The Ct values obtained for the Albumin, Wif1, NPY and PENK genes correspond by default to 100% of methylated copies.

ΔCtₛₐₘₚₗₑ=Ct(targetₛₐₘₚₗₑ)−Ct(referenceₛₐₘₚₗₑ)

ΔCt_{standard}=Ct(target_{standard})−Ct(reference_{standard})

Then ΔΔCt between the standard and the sample is calculated:

ΔΔCt=ΔCt_{standard}−ΔCtₛₐₘₚₗₑ

The method of calculation of the ΔΔCt values assumes that the efficiencies of PCR amplification of the 2 genes are equal to 100%. Thus, in other words, this method of calculation assumes that the concentration of products is doubled at each cycle of the exponential phase of PCR. It is therefore deduced from this that the normalized degree of methylation of a target gene is determined by the formula

2^{-ΔΔCt}=2^{(Ctgene-Ct albumin)standard-(Ctgene-Ctalbumin)sample}

This method gives a relative degree of methylation as a function of the positive control used (standard DNA). Moreover, it takes into account the variations in the copy number of the reference gene used. These variations are necessarily due to the variations in the amount of DNA that was used for carrying out the PCR. Thus, the results are not distorted by the nature of the reference gene.

Preferably, the reference gene is a housekeeping gene. Even more preferably, it is the albumin gene.

Preferably, the method of the invention comprises simultaneous measurement of the degrees of methylation:
of the NPY and PENK genes or respective fragments or variants thereof, and/or
of the NPY and Wif1 genes or respective fragments or variants thereof, and/or
of the PENK and Wif1 genes or respective fragments or variants thereof.

More preferably, the method of the invention comprises simultaneous measurement of the degrees of methylation:
of the sequences SEQ ID No. 1 and SEQ ID No. 2; and/or
of the sequences SEQ ID No. 1 and SEQ ID No. 3; and/or
of the sequences SEQ ID No. 2 and SEQ ID No. 3.

More preferably, the method of the invention comprises simultaneous measurement of the degrees of methylation on the one hand of the Wif1 genes and of a housekeeping gene or respective fragments or variants thereof, and on the other hand of the NPY and PENK genes or respective fragments or variants thereof. Preferably, the housekeeping gene is albumin. The inventors have effectively demonstrated that the application of a multiplex MSP on the Wif1 and albumin genes or fragments or variants thereof on the one hand, and the NPY and PENK genes or fragments or variants thereof on the other hand, increases the sensitivity of the screening test for colorectal cancer.

Thus, simultaneous measurement of the degrees of methylation on two of the genes selected from NPY, PENK, Wif1 and fragments or variants thereof makes it possible to recover subjects detected as negative according to the classical techniques of the prior art and who nevertheless have colorectal cancer.

Preferably, the method of the invention further comprises a step of comparing the degrees of methylation with a threshold value. When the sum of the degrees of methylation of a combination of two genes selected from the group consisting of NPY and PENK or respective fragments or variants thereof, NPY and Wif1 or respective fragments or variants thereof, and PENK and Wif1 or respective fragments or variants thereof, is above this threshold value, the subject from whom the sample tested was obtained is deemed positive for colorectal cancer.

Preferably, the method of the invention further comprises a step of comparing the degrees of methylation on the one hand of the Wif1 genes and of a housekeeping gene, or fragments or variants thereof and on the other hand of the NPY and PENK genes or fragments or variants thereof against a threshold value, said threshold value being specified depending on the requirements in terms of specificity and sensitivity. To obtain an acceptable test, this threshold value is about 2%. Thus, it is necessary to compare on the one hand the degree of methylation of the Wif1 genes and of a housekeeping gene with a threshold value, and on the other hand the degree of methylation of the NPY and PENK genes with said threshold value.

“Sensitivity” means the proportion of positive tests in subjects who have colorectal cancer.

“Specificity” means the proportion of negative tests in subjects who do not have colorectal cancer.

“Cut-off”, or “threshold value”, means the limit value of the degree of methylation of the target genes, above which the sample, and therefore the subject from whom the latter was obtained, is deemed positive for the diagnosis of colorectal cancer according to the method of the invention.

The inventors conducted extensive studies on a group of 246 subjects, 23 of whom had diagnosed colorectal cancer, 44 had an another type of cancer and 179 were healthy.

By applying multiplex genetic testing on combinations of target genes from the subjects' serum, the inventors were able to determine a threshold value, above which the subject is correctly screened as being positive for colorectal cancer, with a given sensitivity and specificity. The sensitivity and the specificity of the screening test are therefore dependent on the threshold value selected. Thus, this threshold value should be adjusted in relation to appropriate sensitivity and specificity. The relevant values of sensitivity and specificity depend on several criteria, generally depending on the population to be diagnosed. Preferably, this threshold value is between 1 and 5%, preferably between 1 and 3.5%, preferably between 1.5 and 3%, preferably between 2 and 3%, and even more preferably this threshold value is 2%.

The inventors have shown that a threshold value of 2% allows the screening test to have a sensitivity of 65.2% and a specificity of 95%. If we lower this threshold value, the sensitivity increases but the specificity decreases. Adopting a step-by-step approach, the inventors succeeded in identifying the most relevant threshold value for developing a test that is sensitive to the genetic marker of colorectal cancer and sufficiently specific to rule out the false positives and false negatives. Thus, depending on the target population and the predisposition of said population to colorectal cancer, the test of the invention can be applied with different threshold values, and thus a modified sensitivity and specificity. In fact, if the target population is a high-risk population in which the subjects are likely to develop colorectal cancer, it is possible to alter the threshold value in order to modulate the sensitivity and specificity of the test.

Preferably, the method of the invention uses a biological sample selected from whole blood, blood plasma, urine and feces of said subject. More preferably, said biological sample is selected from whole blood, plasma and urine of said subject. The method of the invention is therefore very advantageous since it no longer requires carrying out tests on the subject's feces. Thus, the method of the invention is noninvasive and is not prohibitive for a subject requiring a screening test for colorectal cancer.

Preferably, the method of the invention further comprises a step of examination of the colon of said subject. Preferably, this examination step corresponds to a visual colonoscopy. Alternatively, this examination step corresponds to a virtual colonoscopy, also called “coloscanner”. The coloscanner technique is based on examination of the colon by scanner and reconstruction of the internal image of the alimentary canal in 3 dimensions. This colonoscopy makes it possible to verify the presence of colorectal cancer in a subject deemed positive for diagnosis of colorectal cancer according to the method of the invention. Preferably, detection of cancerous tissue is then confirmed by histological examination.

### EXAMPLE

### Selection of the Candidate Genes

### 1. Biological Material and Extraction of Genomic DNA

In a study conducted in humans outside of the screening context, the inventors collected feces, blood, and urine before colonoscopy and extracted the DNA from these effluents. DNA extracted from tissue was also obtained from normal mucosa and pathological mucosa (adenomatous polyps, cancer) either by endoscopic biopsy during the colonoscopy or from the surgical specimen. These subjects were undergoing colonoscopy following specialized consultation (it is therefore a population with functional disorders or anemia without a clear etiology). The aim of this study was to validate a fecal molecular test in comparison with the occult blood test.

In total, 648 patients were included, and the inventors analyzed 577 different samples. Several molecular markers (point mutations, methylation) were investigated. The inventors favored the marker methylation approach and thus adopted a strategy of selecting several genes, which were used for devising a multiplex test (NPY, PENK).

The biological samples (blood, urine, feces), before the colonoscopic examination, and tissue samples (normal mucosa, adenomatous polyps, invasive cancer) were submitted to DNA extraction using the QiAmp midi kit (Qiagen). This was DNA from feces obtained from normal subjects (N=10), and subjects with cancer (N=10).

### 2. Methylation Chip

The “Goldengate” methylation chip, marketed by the company Illumina (USA), can be used for analyzing the methylation of 1505 CpG sites, which correspond to 807 genes, of which 28.6% contain one CpG site per gene, 57.3% contain two CpG sites per gene and 14.1% contain at least three sites. The DNA samples were treated with a solution of sodium bisulfite, using the EZ DNA Methylation kit (ref. D5002, Zymo Research), and then tagged with two fluorescence probes separately targeting the methylated (beta) and unmethylated (alpha) forms of the DNA strands and submitted to gene amplification (PCR). The degree of methylation of the gene under investigation was established based on the value of the ratio (beta)/(alpha+beta) in the range from 0 (unmethylated) to 1 (fully methylated). For each value reported there was a corresponding p value, denoting the probability of a poor estimate of the degree of methylation. Descriptive bioinformatic analysis was carried out based on the threshold value p<0.01.

### 3. Approach for Selecting the Relevant Genes

The approach adopted by the inventors made it possible to identify the relevant targets in different biological effluents (blood, feces, urine) with reference to tissue (normal and tumoral).

Intergroup comparisons were performed with a strategy based on identification of the best intergroup differential markers (normal versus cancers) for tissue or the same biological effluent on the one hand and on the concordance of signals between different biological effluents on the other hand. Out of 1015 genes regarded as being subject to possible methylation, a list of hypermethylated genes (Cancer-Normal> or =20% with a value p<0.0001 in technical terms) was established for the tissue; it contained 146 hypermethylated targets.

In the same way, the list of hypermethylated genes according to the same criterion contained 134 targets in the stool and 122 targets in the blood. Then merging of the lists with one another based on the individual values found 34 common targets between tissue and blood and 46 targets between feces and blood. Finally, the inventors identified the NPY, PENK and Wif1 genes as relevant, and more particularly the fragments of these genes, SEQ ID No. 1, 2 and 3 respectively.

The inventors thus identified 3 candidate genes (Wif1, NPY, and PENK). The inventors demonstrated the advantages of these genes for developing genetic tests based on carrying out multiplex methylation-specific PCR (MSP).

### 4. Gene Amplification in Real Time by the MSP Technique

The TaqMan-MGB primers and probes (Applied Biosystems) were generated taking into account the modification of the DNA. The following table groups together the sequences of the primers and probes corresponding to the “housekeeping” gene: Albumin as well as those of fragments of the 3 genes NPY, PENK and Wif1 (SEQ ID No. 1, 2 and 3 respectively).

The following table gives the nucleotide sequences of the amplification products of the housekeeping gene and of the target genes.

| | Nucleic acid sequences for methylation-specific PCR |
|---|---|
| NPY | Unmodified DNA (SEQ ID No. 1): |
| | 5′ CGCGGCGAGGAAGCTCCATAAAAGCCCTGTCGCGACCCGCTCTCTGCACCCCATCCG |
| | CTGGCTCTCACCCCTCGGAGACGCTCGCCCGACAGCAT 3′ |
| | Modified DNA: Amplicon with 95 bases (SEQ ID No. 5) |
| | 5′ CGCGGCGAGGAAGTTTTATAAAAGTTTTGTCGCGATTCGTTTTTTGTATTTTATTCG |
| | TTGGTTTTTATTTTTCGGAGACGTTCGTTCGATAGTAT 3′ |
| PENK | Unmodified DNA (SEQ ID No. 2): |
| | 5′ CGGGTAGACGTTTTTAAATTCGTTTAGTAAAGATAAGTTTCGGGTAAATTTATTTAC |
| | GTTGACGTTGTTCGGATGGAGTAGGTTAATTGGAAAAGTCGGGTTTAGATACG 3′ |
| | Modified DNA: Amplicon with 110 bases (SEQ ID No. 6) |
| | 5′ CGGGTAGACGTTTTTAAATTCGTTTAGTAAAGATAAGTTTCGGGTAAATTTATTTAC |
| | GTTGACGTTGTTCGGATGGAGTAGGTTAATTGGAAAAGTCGGGTTTAGATACG 3′ |
| Wif1 | Unmodified DNA (SEQ ID No. 3): |
| | 5′ TCTGACGGCGCCAGGTTGCGTAGGTGCGGCACGAGGAGTTTTCCCGGCAGCGAGGA |
| | GGTCCTGAGCAGC 3′ |
| | Modified DNA: Amplicon with 69 bases (SEQ ID No. 7) |
| | 5′ TTTGACGGCGTTAGGTTGCGTAGGTGCGGTACGAGGAGTTTTTTCGGTAGCGAGGA |
| | GGTTTTGAGTAGT 3 |
| Albumin | Unmodified DNA (SEQ ID No. 4): |
| | 5′ GGGATGGAAAGAATCCTATGCCTGGTGAAGGTCAAGGGTTCTCATAACCTACAGA |
| | GAATTTGGGGTCAGCCTGTCC 3′ |
| | Modified DNA: Amplicon with 77 bases (SEQ ID No. 8) |
| | 5′ GGGATGGAAAGAATTTTATGTTTGGTGAAGGTTAAGGGTTTTTATAATTTATAGAG |
| | AATTTGGGGTTAGTTTGTTT 3′ |

The following table gives the sequences of the sense primers, antisense primers and TaqMan probes used.

| | Sense Primer | Antisense Primer | TaqMan-MGB Probe |
|---|---|---|---|
| Wif1 | SEQ ID No. 9 | SEQ ID No. 10 | SEQ ID No. 11 |
| | TTTGACGGCGTTAGGTTGC | ACTACTCAAAACCTCCTCGCTACC | CGGTACGAGGAGTTTT |
| | T° m = 58.4° C. 19 bases | T° m = 58.1° C. 24 bases | T° m = 70° C 16 bases |
| | 3 CpG | 2 CpG | 2 CpG |
| NPY | SEQ ID No. 12 | SEQ ID No. 13 | SEQ ID No. 14 |
| | CGCGGCGAGGAAGTTTTATA | ATACTATCGAACGAACGTCTCCG | CGCGATTCGTTTTTTGTA |
| | T° m = 59° C. 20 bases | T° m = 58.4° C. 23 bases | T° m = 68° C. 18 bases |
| | 3 CpG | 4 CpG | 3 CpG |
| PENK | SEQ ID No. 15 | SEQ ID No. 16 | SEQ ID No. 17 |
| | CGGGTAGACGTTTTTAAATTC | CGTATCTAAACCCGACTTTTC | CGTTGACGTTGTTCGGAT |
| | T° m = 52.3° C. 21 bases | T° m = 52.2° C. 21 bases | T° m = 70° C. 18 bases |
| | 3 CpG | 2 CpG | 3 CpG |
| Albumin | SEQ ID No. 18 | SEQ ID No. 19 | SEQ ID No. 20 |
| | GGGATGGAAAGAATTTTATGTT | AAACAAACTAACCCCAAATTCT | AGGGTTTTTATAATTTA |
| | T° m = 52.9° C. 22 bases | T° m = 52.7° C. 22 bases | T° m = 63° C. 17 bases |
| | 0 CpG | 0 CpG | 0 CpG |

Amplification is performed in 96-well plates sealed with an optical-grade film, qPCR compatible (ABgene). 5.8 μl of modified DNA (10 ng standard DNA and DNA from patients) is added at a final concentration of 400 nmol of each primer (sense and antisense) and at a final concentration of 250 nmol of TaqMan-MGB probe and 1× of PCR solution (Applied Biosystems). The reaction volume is 20 μl. The amplification conditions are as follows:
Hot start: 94° C. for 15 minutes;
Up to 48 cycles: 94° C. for 15 seconds, denaturing step;
60° C. for 60 seconds, hybridization (annealing) and elongation steps.
   The standard DNA is a DNA that is universally methylated (ref. D5011, Zymo Research). Following modification using the EZ DNA Methylation kit (ref. D5002, Zymo Research), it is used in MSP as reference. The Ct values obtained on the fragments of the Albumin, Wif1, NPY and PENK genes correspond by default to 100% of methylated copies.

a) Percentage Methylation According to the Delta-Delta Ct Method

The degree of methylation is quantified according to the method of ΔΔCt=2^{(Ctgene-Ctalbumin)sample+(Ctgene-Ctalbumin)standard}. This method gives a relative value of the degree of methylation as a function of the positive control used (standard DNA). It takes into account the variations in the copy number of the Albumin gene that can only be due to a variation in amounts of DNA used for carrying out MSP.

b) Evaluation Phase of the MSPM Molecular Test on Tissue DNA

The evaluation phase was carried out using 32 human tissue DNAs including 16 tumoral DNAs and 16 so-called “normal” autologous tissue DNAs.

Cut-off = 20%

| | Sensitivity | Specificity |
|---|---|---|
| Monoplex MSP | | |
| Wif1 | 68.75% | 100% |
| NPY | 100% | 93.75% |
| PENK | 75% | 93.75% |
| Multiplex MSP | | |
| NPY + PENK (duplex) | 100% | 93.75% |
| Wif1 + PENK (duplex) | 75% | 100% |
| Wif1 + NPY (duplex) | 100% | 100% |
| Combination of multiplex | | |
| MSP = 2 duplexes per run | | |
| Wif1 + albumin | 100% | 100% |
| NPY + PENK | | |

c) Determination of the Performance of the Genetic Test on Serum DNA by “Methylation-Specific PCR”

The inventors developed a multiplex MSP based on tissue DNA. The very good performance of the multiplex test obtained in terms of specificity and sensitivity on tissue samples led to undertaking validation for serum samples.

i. Biological Material

We evaluated the suitability of the multiplex genetic test on samples of 246 sera obtained from hospitalized patients who underwent the multiplex test protocol.

ii. Extraction of the Serum DNA: Choice of Extraction Kit

The inventors evaluated the suitability of the DNA extraction kit marketed by the company Qiagen, QiAmp midi kit, and the ZR Serum Kit (ref. D3013) marketed by the company Zymo Research. The ZR Serum Kit is the one that was adopted. A volume of 4 ml of serum (8-10 ml of whole blood) is recommended for obtaining a sufficient quantity of nucleic acid molecules necessary for application of 4 MSP, corresponding to 2 duplexes performed in combination [(Wif1+Albumin) and (NPY+PENK)].

d) Validation

The validation phase of the multiplex test was conducted on a first sampling of 246 sera. Among these 246 sera, 23 were from individuals with cancer of the colon or rectum, 44 from individuals with another type of cancer, and 179 from individuals without cancer. The performance, given in terms of Specificity and Sensitivity, of multiplex MSP targeting the 3 candidate genes in combination (2 MSP duplexes) are shown in the following table. Duplex 1 corresponds to multiplex MSP of Wif1 and of albumin whereas duplex 2 corresponds to multiplex MSP of NPY and of PENK.

| Multiplex genetic | Cut-off = 3% | | Cut-off = 2% | |
|---|---|---|---|---|
| test on serum | Sensitivity | Specificity | Sensitivity | Specificity |
| 246 sera comprising: | 56.5% | 98.3% | 65.2% | 95% |
| 23 colorectal cancers | | | | |
| 44 other cancers | | | | |
| 179 non-cancers | | | | |

It appears that the specificity and sensitivity depend on the threshold value selected initially. Thus, a threshold value (or cut-off) of 3% gives a sensitivity of 56.5% and a specificity of 98.3%, whereas a threshold value of 2% gives a sensitivity of 65.2% and a specificity of 95%. Thus, the threshold value should be adjusted according to the population of individuals to be diagnosed.

e) Comparison of Performance: Multiplex Test Versus Monoplex Test

The phase of validation of the multiplex test according to the invention determined the performance of this test in terms of Sensitivity (65.2%) (15 colorectal cancers CRC detected out of a total of 23) and 95% in terms of Specificity. The following table shows the results of the 15 individuals with colorectal cancer and positive in multiplex mode and in multiplex combination mode, compared with the results obtained in monoplex mode.

| | | | | Multiplex MSP (Duplex) | | | Combination of |
|---|---|---|---|---|---|---|---|
| | | | | Cut-off = 2% | | | multiplex MSP |
| | Monoplex MSP | | | NPY + | NPY + | PENK + | (duplex 1 + |
| ID | NPY | PENK | Wif1 | PENK | Wif1 | Wif1 | duplex 2) |
| CCR1 | 65.33 | 11.35 | 12.79 | + | + | + | + |
| | (+) | (+) | (+) | | | | |
| CCR2 | 3.16 | 2.84 | 5.57 | + | + | + | + |
| | (+) | (+) | (+) | | | | |
| CCR3 | 38.03 | 4.40 | 100.63 | + | + | + | + |
| | (+) | (+) | (+) | | | | |
| CCR4 | 46.37 | 9.89 | 15.19 | + | + | + | + |
| | (+) | (+) | (+) | | | | |
| CCR5 | 0 | 0.12 | 2.72 | − | + | + | + |
| | (−) | (−) | (+) | | | | |
| CCR6 | 56.07 | 105.85 | 0.07 | + | + | + | + |
| | (+) | (+) | (−) | | | | |
| CCR7 | 1.59 | 1.14 | 0.87 | + | + | + | + |
| | (−) | (−) | (−) | | | | |
| CCR8 | 40.55 | 2.68 | 19.57 | + | + | + | + |
| | (+) | (+) | (+) | | | | |
| CCR9 | 20.92 | 2.3 | 6.37 | + | + | + | + |
| | (+) | (+) | (+) | | | | |
| CCR10 | 54.33 | 8.87 | 39.24 | + | + | + | + |
| | (+) | (+) | (+) | | | | |
| CCR11 | 0 | 0 | 4.40 | − | + | + | + |
| | (−) | (−) | (+) | | | | |
| CCR12 | 7.22 | 0 | 1.92 | + | + | − | + |
| | (+) | (−) | (−) | | | | |
| CCR13 | 2.31 | 0.59 | 6.13 | + | + | + | + |
| | (+) | (−) | (+) | | | | |
| CCR14 | 4.44 | 0 | 0 | + | + | − | + |
| | (+) | (−) | (−) | | | | |
| CCR15 | 1.43 | 0.81 | 0.25 | + | − | − | + |
| | (−) | (−) | (−) | | | | |

Duplex 1 corresponds to a multiplex MSP of the fragments of Wif1 and of albumin, whereas duplex 2 corresponds to a multiplex MSP of the fragments of NPY and of PENK.
The inventors have thus demonstrated that carrying out multiplex (duplex) MSP increases the sensitivity value of the test (recovery of subjects with colorectal cancer classified as negative in monoplex MSP).
The inventors have also demonstrated that the combination of multiplex MSP (notably the combination of duplex 1 with duplex 2) offers even better results. The 15 individuals with colorectal cancer are all positive in the test.

1. A method of screening for colorectal cancer in a subject, comprising a step of measuring the degree of methylation of at least one of the genes selected from NPY, PENK and fragments or variants thereof in a biological sample obtained from said subject.

2. The method as claimed in claim 1, characterized in that said fragments are selected from the sequences SEQ ID No. 1 and SEQ ID No. 2.

3. The method as claimed in claim 1 or 2, characterized in that it further comprises a step of measuring the degree of methylation of the Wif1 gene or of fragments or variants thereof in a biological sample obtained from said subject.

4. The method as claimed in claim 3, characterized in that said fragment is the sequence SEQ ID No. 3.

5. The method as claimed in any one of claims 1 to 4, characterized in that it further comprises a step of measuring the degree of methylation of a housekeeping gene, preferably the albumin gene or a fragment or variant thereof.

6. The method as claimed in claim 5, characterized in that said fragment is the sequence SEQ ID No. 4.

7. The method as claimed in any one of claims 1 to 6, characterized in that it comprises simultaneous measurement of the degrees of methylation:
of the NPY and PENK genes or respective fragments or variants thereof, and/or
of the NPY and Wif1 genes or respective fragments or variants thereof, and/or
of the PENK and Wif1 genes or respective fragments or variants thereof.

8. The method as claimed in any one of claims 1 to 7, characterized in that it comprises simultaneous measurement of the degrees of methylation:
of the sequences SEQ ID No. 1 and SEQ ID No. 2; and/or
of the sequences SEQ ID No. 1 and SEQ ID No. 3; and/or
of the sequences SEQ ID No. 2 and SEQ ID No. 3.

9. The method as claimed in any one of claims 1 to 8, characterized in that it comprises simultaneous measurement of the degrees of methylation on the one hand of the Wif1 genes and a housekeeping gene or respective fragments or variants thereof, and on the other hand of the NPY and PENK genes or respective fragments or variants thereof.

10. The method as claimed in claim 9, characterized in that it further comprises a step of comparing said degrees of methylation with a threshold value, said threshold value being between 1.5 and 3%.

11. The method as claimed in claim 10, characterized in that said threshold value is 2%.

12. The method as claimed in any one of claims 1 to 11, characterized in that said biological sample is selected from whole blood, blood plasma, urine and feces from said subject.

13. The method as claimed in any one of claims 1 to 12, characterized in that said biological sample is selected from whole blood, plasma and urine from said subject.

14. The method as claimed in any one of claims 1 to 13, characterized in that it further comprises a step of examination of the colon of said subject, followed by a step of histological examination.


SEQUENCE LISTING (20 sequences)

SEQ ID NO: 1 - DNA, 95 bp, Homo sapiens
cgcggcgaggaagctccataaaagccctgtcgcgacccgctctctgcaccccatccgctggctctcacccctcggagacgctcgcccgacagcat

SEQ ID NO: 2 - DNA, 110 bp, Homo sapiens
cgggtagacgtttttaaattcgtttagtaaagataagtttcgggtaaatttatttacgttgacgttgttcggatggagtaggttaattggaaaagtcgggtttagatacg

SEQ ID NO: 3 - DNA, 69 bp, Homo sapiens
tctgacggcgccaggttgcgtaggtgcggcacgaggagttttcccggcagcgaggaggtcctgagcagc

SEQ ID NO: 4 - DNA, 76 bp, Homo sapiens
gggatggaaagaatcctatgcctggtgaaggtcaagggttctcataacctacagagaatttggggtcagcctgtcc

SEQ ID NO: 5 - DNA, 95 bp, Homo sapiens
cgcggcgaggaagttttataaaagttttgtcgcgattcgttttttgtattttattcgttggtttttatttttcggagacgttcgttcgatagtat

SEQ ID NO: 6 - DNA, 110 bp, Homo sapiens; identical to SEQ ID NO: 2

SEQ ID NO: 7 - DNA, 69 bp, Homo sapiens
tttgacggcgttaggttgcgtaggtgcggtacgaggagttttttcggtagcgaggaggttttgagtagt

SEQ ID NO: 8 - DNA, 76 bp, Homo sapiens
gggatggaaagaattttatgtttggtgaaggttaagggtttttataatttatagagaatttggggttagtttgttt

SEQ ID NO: 9 - DNA, 19 bp, Artificial
  Amorce Sens
tttgacggcgttaggttgc

SEQ ID NO: 10 - DNA, 24 bp, Artificial
  Amorce antisens
actactcaaaacctcctcgctacc

SEQ ID NO: 11 - DNA, 16 bp, Artificial
  Sonde TaqMan
cggtacgaggagtttt

SEQ ID NO: 12 - DNA, 20 bp, Artificial
  Amorce sens
cgcggcgaggaagttttata

SEQ ID NO: 13 - DNA, 23 bp, Artificial
  Amorce antisens
atactatcgaacgaacgtctccg

SEQ ID NO: 14 - DNA, 18 bp, Artificial
  Sonde TaqMan
cgcgattcgttttttgta

SEQ ID NO: 15 - DNA, 21 bp, Artificial
  Amorce sens
cgggtagacgtttttaaattc

SEQ ID NO: 16 - DNA, 21 bp, Artificial
  Amorce antisens
cgtatctaaacccgacttttc

SEQ ID NO: 17 - DNA, 18 bp, Artificial
  Sonde TaqMan
cgttgacgttgttcggat

SEQ ID NO: 18 - DNA, 22 bp, Artificial
  Sonde amorce
gggatggaaagaattttatgtt

SEQ ID NO: 19 - DNA, 22 bp, Artificial
  Amorce antisens
aaacaaactaaccccaaattct

SEQ ID NO: 20 - DNA, 17 bp, Artificial
  Sonde TaqMan
agggtttttataattta